(19) United States
(12) Patent Application Publication
(10) Pub. No.: US 2011/0287068 A1
(43) Pub. Date: Nov. 24, 2011

(54) FIBRIN AND FIBRINOGEN MATRICES AND USES OF SAME

(75) Inventors: Sandu Pitaru, Ramat-Gan (IL); Naphtali Savion, Givat Shmuel (IL)

(73) Assignee: Ramot at Tel-Aviv University Ltd., Tel-Aviv (IL)

(21) Appl. No.: 13/143,196

(22) PCT Filed: Dec. 31, 2009
(86) PCT No.: PCT/IL09/01237

§ 371 Date: Jul. 4, 2011

Related U.S. Application Data
(60) Provisional application No. 61/193,872, filed on Jan. 2, 2009.

(51) Int. Cl. A61K 9/00; C07K 1/107; C07K 14/745; A61K 8/64; A61P 17/02; A61P 43/00; C07K 14/75; A61K 38/36

(52) U.S. Cl. 424/400; 530/382; 514/15.3; 514/13.6

(57) ABSTRACT

There is provided compositions-of-matter comprising fibrin or fibrinogen crosslinked with at least one reducing sugar.

### FIELD AND BACKGROUND OF THE INVENTION

The present invention, in some embodiments thereof, relates to a crosslinked protein, and more particularly, but not exclusively, to crosslinked proteins such as fibrin and fibrinogen, to processes of preparing same and to uses thereof.

The progress made in the purification of extracellular matrix proteins and in the production of recombinant proteins has enabled their utilization for therapeutic purposes. During the past decade there has been a continuous increase in the discovery of proteins that serve as new biological drugs. There has also been an increase in the utilization of matrix proteins for tissue engineering and regenerative medicine. The efficiency of these therapeutic proteins depends to a great extent on their stability and resistance to degradation following their administration.

Protein stability is of paramount importance for the development, homeostasis and repair of any organism. The stability of proteins is tightly regulated within the cell and in the extracellular compartment. In the extracellular compartment, protein stability, and consequently the longevity of the protein functionality, is controlled by protein stabilizing enzymes, by the availability and activity of degradation enzymes and by inhibitors for these enzymes.

Fibrin is an insoluble biopolymer formed by the polymerization of fibrinogen. Fibrinogen is produced by the liver, and circulates in the blood as a plasma glycoprotein at a concentration of 2.5 grams/liter.

Fibrinogen is composed of 3 polypeptides chains: Aα, Bβ and γ. A and B are fibrinopeptides that are cleaved by thrombin from the Aα and Bβ chains. This cleavage results in the formation of fibrin molecules that undergo conformational changes which expose polymerization sites. Subsequently, fibrin molecules polymerize into a 3-dimensional hydrogel consisting of fibrin fibers and a physiological liquid. Fibrinogen cleavage and fibrin polymerization occurs under physiologic conditions and particularly during bleeding. Following polymerization, the fibrin molecules within the fibers are crosslinked by the plasma enzyme transglutaminase (factor XIIIa) Crosslinking confers mechanical strength and proteolytic resistance to the fibrin scaffold. Fibrin polymerization and the formation of a fibrin scaffold are central parts in the haemostatic process and in the initiation of wound healing [Mosesson et al., Ann NY Acad Sci 2001, 936:11-30].

Fibrin plays an important role in the process of hemostasis and wound healing. Fibrin also plays important roles in cell-matrix interactions, in inflammation and in neoplasia. The various biological properties of fibrinogen and fibrin are extensively reviewed by Weisel [Adv Protein Chem 2005, 70:247-299] and Mosesson et al. [Ann NY Acad Sci 2001, 936:11-30].

Fibrinogen and fibrin bind several attachment proteins (fibronectin, thrombospondin, fibulin-1, von Willebrand factor), growth factors (fibroblast growth factor-2, vascular endothelial growth factor), cytokines (interleukin 1β), and albumin, all of which are important in initiating platelet adhesion to the fibrin fibers, chemotaxis of macrophages and fibroblasts, angiogenesis, and cell proliferation [Weisel, Adv Protein Chem 2005, 70:247-299].

In wound healing, fibrin clots function as a provisional matrix that is later replaced by cells and matrix of the healing tissues. Replacement of the fibrin clot involves the degradation of the fibrin scaffold (fibrinolysis) by matrix metalloproteinases, and mainly by plasmin which is derived from plasminogen following the cleavage of the latter by tissue plasminogen activator (t-PA). In vivo, fibrinolysis is inhibited by a number of plasma proteins, such as α2-macroglubulin, α2-antiplasmin, plasminogen inhibitor type 1, apolipoprotein (a) and others [Weisel, Adv Protein Chem 2005, 70:247-299; Mosesson et al. Ann NY Acad Sci 2001, 936:11-30; Mosesson, J Thromb Haemost 2005, 3:1894-1904].

The process of fibrinogen polymerization and the formation of a fibrin scaffold can be reproduced in vitro by cleaving the fibrinopeptides A and B from purified fibrinogen with thrombin. The size of the fibrin fibers and the scaffold porosity, and consequently the biochemical and mechanical properties of the scaffold, can be modified by changing the fibrinogen concentration, the fibrinogen-thrombin ratio, and the ionic strength [Carr & Hermans, Macromolecules 1978, 11:46-50].

The role of fibrin in wound healing, its biological properties, and the fact that fibrinogen can be easily isolated from plasma and polymerized in vitro, have led to the use of fibrin in applications such as fibrin glues for homeostasis, and fibrin scaffolds for cardiac, cartilage, bone and skin repair [Ahmed et al., Tissue Eng Part B Rev 2008, 14:199-215; MacGillivray, J Card Surg 2003, 18:480-485].

The fibrin molecule may be modified with a synthetic molecule such as polyethylene glycol, a process that modifies the biological properties of the fibrin [Ahmed et al., Tissue Eng Part B Rev 2008, 14:199-215], and by the utilization of non-enzymatic crosslinking agents such as genepin [Dare et al., Cells Tissues Organs 2009, 190:313-325], or the synthetic fixative glutaraldehyde [Ahmed et al., Tissue Eng Part B Rev 2008, 14:199-215].

Tanaka et al. [J Biol Chem 1988, 263:17650-17657] teaches that sugars induce crosslinking of collagen scaffolds, rendering them more resistant to enzymatic degradation by metalloproteinases, and that D-ribose is more effective than glucose at inducing crosslinking.

U.S. Pat. No. 4,971,954 teaches cross-linked collagen type-1-based matrices, prepared by cross-linking native collagen polypeptide chains, using D-ribose as a crosslinking agent.

U.S. Pat. No. 5,955,438 teaches a matrix of atelocollagen type I fibrils crosslinked by a reducing sugar, and a process of preparing same by incubating collagen type I with pepsin, dissolving the resulting atelocollagen and forming a compressed membrane, and reacting the compressed membrane with a reducing sugar.

U.S. Pat. No. 6,682,760 teaches a process for crosslinking atelocollagen type I, by incubating collagen in a solution comprising water, a polar solvent, and a sugar.

### SUMMARY OF THE INVENTION

The prior art teaches various processes of preparing cross-linked collagen, with a reducing sugar as a cross-linking agent.

Fibrin is a fibrillar protein, which, in its native form, is insoluble in aqueous media.

Fibrin scaffold is formed both in vivo (in plasma) and in vitro (in plasma or buffer solution) by a similar process, utilizing similar precursor molecules, specific cleavage of the fibrinogen precursor chains by thrombin, and the following self-assembly of the monomer chains to form fibrillar fibrin. Thus, fibrin that is formed in vitro is substantially identical to fibrin which is naturally formed in vivo.

Fibrinogen, the native precursor of fibrin, is naturally soluble in aqueous media. Fibrinogen is not a fibrillar protein but is rather naturally present as a molecular protein which is soluble in aqueous media.

The present inventors have now uncovered that under certain conditions, cross-linking of fibrin by sugars can be effected. Moreover, the present inventors have surprisingly uncovered that cross-linking by sugars can be effected also with non-fibrillar proteins such as fibrinogen.

In the case of fibrinogen, cross-linking is effected upon converting the fibrinogen into a precipitated amorphous structure (as opposed to fibrillar structure).

Accordingly, novel crosslinked proteins (e.g., fibrin, fibrinogen) are described herein. The crosslinking is effected by a non-enzymatic process and utilizes non-toxic sugars and non-toxic solvents such as ethanol.

According to an aspect of some embodiments of the present invention there is provided a composition-of-matter comprising fibrinogen being crosslinked with at least one reducing sugar.

According to an aspect of some embodiments of the present invention there is provided a composition-of-matter comprising fibrin being crosslinked with at least one reducing sugar.

According to an aspect of some embodiments of the present invention there is provided a process for producing a composition-of-matter comprising fibrinogen being crosslinked with at least one reducing sugar, the process comprising reacting fibrinogen with the at least one reducing sugar in a crosslinking solution which comprises the reducing sugar and a polar organic solvent.

According to an aspect of some embodiments of the present invention there is provided a process for producing a composition-of-matter comprising fibrin being crosslinked with at least one reducing sugar, the process comprising reacting fibrin with the at least one reducing sugar in a crosslinking solution which comprises the reducing sugar and a polar organic solvent.

According to an aspect of some embodiments of the present invention there is provided a composition-of-matter obtainable by a process described herein.

According to an aspect of some embodiments of the present invention there is provided a pharmaceutical, cosmetic or cosmeceutical composition comprising a composition-of-matter described herein and a pharmaceutically, cosmetically or cosmeceutically acceptable carrier.

According to an aspect of some embodiments of the present invention there is provided a use of a composition-of-matter described herein in the manufacture of a medicament for the treatment of a medical disorder or a cosmetic disorder characterized by a tissue damage.

According to an aspect of some embodiments of the present invention there is provided a method of treating a medical disorder or a cosmetic disorder characterized by a tissue damage in a subject in need thereof, the method comprising administering to the subject a therapeutically effective amount of a composition-of-matter described herein.

According to an aspect of some embodiments of the present invention there is provided a method of performing a procedure selected from the group consisting of tissue regeneration, wound healing, tissue engineering, drug delivery and tissue augmentation in a subject in need thereof, the method comprising administering to the subject a composition-of-matter described herein.

According to an aspect of some embodiments of the present invention there is provided a medical device composed of, or comprising, a composition-of-matter described herein.

According to an aspect of some embodiments of the present invention there is provided a kit for generating a composition-of-matter comprising fibrinogen described herein, the kit comprising
(i) fibrinogen; and
(ii) a reducing sugar.

According to an aspect of some embodiments of the present invention there is provided a kit for generating a composition-of-matter comprising crosslinked fibrin, the kit comprising:
(i) fibrinogen;
(ii) thrombin; and
(iii) a reducing sugar.

According to some embodiments of the invention, the reducing sugar is a pentose.

According to some embodiments of the invention, the pentose is ribose.

According to some embodiments of the invention, the composition-of-matter is characterized by a structure comprising an aggregation of microparticles.

According to some embodiments of the invention, the composition-of-matter is characterized by a fibrillar structure.

According to some embodiments of the invention, the composition-of-matter is in an injectable form.

According to some embodiments of the invention, the composition-of-matter has a predetermined resistance to proteolytic degradation, such that a degradation time of the composition-of-matter when subjected to 1000 units/ml trypsin at 37° C. is selected from a range of 1 hour to 7 days.

According to some embodiments of the invention, the composition-of-matter has a concentration of fibrinogen in a range of 1 mg/ml to 50 mg/ml.

According to some embodiments of the invention, the composition-of-matter has a concentration of fibrinogen in a range of 5 mg/ml to 25 mg/ml. According to some embodiments of the invention, a concentration of the reducing sugar in the crosslinking solution is in a range of 0.1% to 6%.

According to some embodiments of the invention, a concentration of the reducing sugar in the crosslinking solution is in a range of 0.5% to 4%.

According to some embodiments of the invention, a concentration of the reducing sugar in the crosslinking solution is in a range of 0.1% to 2%.

According to some embodiments of the invention, a concentration of the reducing sugar in the crosslinking solution is in a range of 1% to 2%.

According to some embodiments of the invention, fibrinogen is incubated in the crosslinking solution for a period of time in a range of 1 day to 20 days.

According to some embodiments of the invention, the fibrinogen is insoluble in the polar organic solvent, and the process further comprises precipitating the fibrinogen in a solution comprising said polar organic solvent.

According to some embodiments of the invention, the polar organic solvent is a protic solvent.

According to some embodiments of the invention, the polar organic solvent is ethanol.

According to some embodiments of the invention, a concentration of polar organic solvent in the crosslinking solution is in a range of 50% to 100% per volume of the crosslinking solution.

According to some embodiments of the invention, a concentration of polar organic solvent in the crosslinking solution is about 70% per volume of the crosslinking solution.

According to some embodiments of the invention, a concentration of polar organic solvent in the crosslinking solution is at least 80% per volume of the crosslinking solution.

According to some embodiments of the invention, the process further comprises drying the composition-of-matter.

According to some embodiments of the invention, the process further comprises converting the composition-of-matter to an injectable form, the converting comprising particulation of the composition-of-matter into particles of a size sufficiently small so as to be suitable for injection.

According to some embodiments of the invention, the particulation comprises passing the composition-of-matter through a needle.

According to some embodiments of the invention, the composition-of-matter further comprises a pharmaceutically active agent being contained within the composition-of-matter or on a surface of the composition-of-matter.

According to some embodiments of the invention, the pharmaceutically active agent is selected from the group consisting of a therapeutically active agent and a labeling agent.

According to some embodiments of the invention, the therapeutically active agent is selected from the group consisting of a stem cell, a growth factor, a bone morphogenetic protein, a cell, a cytokine, a hormone, a medicament, a mineral, a plasmid with therapeutic potential, and a combination of thereof.

According to some embodiments of the invention, the composition-of-matter is identified for use in the treatment of a medical disorder or a cosmetic disorder characterized by a tissue damage.

According to some embodiments of the invention, the disorder is treatable by a procedure selected from the group consisting of tissue regeneration, wound healing, tissue engineering, drug delivery, and tissue augmentation.

According to some embodiments of the invention, the composition-of-matter is administered to the subject by implantation.

According to some embodiments of the invention, the composition-of-matter is administered to the subject by injection.

According to some embodiments of the invention, the device is in the form of a membrane.

According to some embodiments of the invention, the fibrinogen and the reducing sugar are each packaged individually in the kit.

According to some embodiments of the invention, the fibrinogen and the reducing sugar are packaged together in the kit.

According to some embodiments of the invention, the kit further comprises a polar organic solvent described herein.

According to some embodiments of the invention, the composition-of-matter has an optical density at least 20% higher than that of non-crosslinked fibrin.

According to some embodiments of the invention, the composition-of-matter exhibits a resistance to proteolytic degradation which is at least 20% higher than that of Factor XIIIa-crosslinked fibrin.

According to some embodiments of the invention, the composition-of-matter has a concentration of fibrin in a range of 3 mg/ml to 200 mg/ml.

According to some embodiments of the invention, the composition-of-matter has a concentration of fibrin in a range of 25 mg/ml to 100 mg/ml. According to some embodiments of the invention, fibrin is incubated in the crosslinking solution for a period of time in a range of 1 day to 20 days.

According to some embodiments of the invention, the process further comprises, prior to reacting fibrin with the reducing sugar, reacting fibrinogen with thrombin so as to obtain fibrin.

According to some embodiments of the invention, the fibrin, the thrombin and the reducing sugar are each packaged individually in the kit.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a graph showing the percentage of crosslinked fibrinogen (injectable form) that was degraded following incubation for 6 hours with various concentrations (in units/ml) of trypsin; fibrinogen was crosslinked by incubation in crosslinking solution for 5 days (CL 5d) or for 11 days (CL 11d);

FIG. 2 is a graph showing the percentage of crosslinked fibrinogen (injectable form) that was degraded following incubation for various times with 1000 units/ml trypsin; fibrinogen was crosslinked by incubation in crosslinking solution for 5 days (CL 5d) or for 11 days (CL 11d);

FIG. 3 is a graph showing the percentage of crosslinked fibrin (membrane) that was degraded following incubation for various times with 0.25% trypsin; fibrin was crosslinked by incubation in crosslinking solution for 3 days (cl-3days), 6 days (cl-6days) or for 11 days (CL 11days), or was a non-crosslinked control fibrin membrane (non-cl);

FIG. 4 is a graph showing the optical density (O.D.) of fibrin matrices following incubation for various time periods in a crosslinking solution, compared with non cross-linked fibrin matrices (control);

FIG. 5 is a graph showing the optical density (O.D.) of fibrin matrices following incubation for various time periods in a crosslinking solution containing 0.1%, 0.25%, 0.5%, 1% or 2% ribose, or in a control solution containing no ribose (con);

FIG. 6 is a graph showing the optical density (O.D.) of fibrin matrices following incubation for various time periods in a crosslinking solution containing 50%, 70%, 90% or 100% ethanol as organic solvent (O.S.), or in a control solution containing no ethanol (con);

FIG. 7 is a graph showing the optical density (O.D.) of fibrin matrices comprising 25 mg/ml fibrin (f25), 50 mg/ml fibrin (f50) or 100 mg/ml fibrin (f100), following incubation for various time periods in a crosslinking solution containing ribose and 90% ethanol (R+(alc90%)), or in a control solution containing no ribose (con);

FIG. 8 is a graph showing the percentage of crosslinked and non-crosslinked fibrin matrices that were degraded following incubation for various times with 0.25% trypsin;

FIGS. 9A and 9B are electron scanning micrographs showing crosslinked fibrin (injectable form) according to embodiments of the present invention, at magnifications of 3,000 (FIG. 9A) and 12,000 (FIG. 9B);

FIGS. 10A and 10B are electron scanning micrographs showing crosslinked fibrinogen (injectable form) according to embodiments of the present invention, at magnifications of 12,000 (FIG. 10A) and 24,000 (FIG. 10B);

FIG. 11 is a graph showing the percentage of fibrin that was degraded following incubation for various times with trypsin; fibrin was crosslinked by incubation in a solution comprising ethanol and ribose (cl-F10) or by transglutaminase, or was a non-crosslinked fibrin control (non cl);

FIG. 12 is a graph showing cell adhesion to crosslinked fibrin membrane surfaces, non-crosslinked fibrin membrane surfaces, and polystyrene surfaces (control), in the presence of, and in the absence of fetal calf serum; and

FIG. 13 is a graph showing cell density (cells/mm²) on crosslinked fibrin membrane surfaces and on non-crosslinked fibrin membrane surfaces (control), 1 day and 3 days following plating of cells.

### DESCRIPTION OF SPECIFIC EMBODIMENTS OF THE INVENTION

The present invention, in some embodiments thereof, relates to a crosslinked protein, and more particularly, to crosslinked fibrin and fibrinogen, to processes of preparing same and to uses thereof.

The principles and operation of the present invention may be better understood with reference to the figures and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Fibrin is a fibrillar protein, as defined herein, which plays an important role in various biological processes, such as homeostasis and wound healing. Fibrinogen is a soluble, non-fibrillar protein, which is the native precursor of fibrin.

The biological properties of fibrin and the fact that fibrinogen can be easily isolated from plasma and polymerized in vitro, have led to the use of fibrin in various applications. A major drawback of in vitro-produced fibrin scaffolds is their relatively fast and uncontrolled fibrinolysis in vitro and in vivo (via specific or non-specific enzymes). The use of synthetic crosslinking agents (e.g., glutaraldehyde) to inhibit fibrinolysis is problematic, as such crosslinking agents are toxic and the concentration at which they may be used is therefore limited. When low concentrations are used, the efficiency of the crosslinking process and consequently the resistance to fibrinolysis is also limited.

The present inventors have now uncovered that under certain conditions, cross-linking of fibrin by sugars can be effected. Moreover, the present inventors have surprisingly uncovered that cross-linking by sugars can be effected also with non-fibrillar proteins such as fibrinogen. The present inventors have further uncovered that by controlling the concentrations of the reducing sugar (e.g., ribose) and the polar solvent (e.g., ethanol) and the incubation time, it is possible to precisely control the stability of both insoluble, cross-linked fibrinogen and fibrin and their resistance to degradation by proteolytic enzymes. Thus, for example, it has been uncovered that the stability of fibrin to proteolytic degradation is increased more than 15-fold compared to fibrin incubated in a solution without a reducing sugar or untreated fibrin.

The present inventors have further uncovered that an organic polar solvent can be used to form a fibrinogen amorphous precipitate. In the presence of a reducing sugar, this fibrinogen precipitate in organic polar solvent results in the formation of intermolecular crosslinking bridges, thereby forming a novel cross-linked fibrinogen matrix which is insoluble in aqueous solution.

It was thus demonstrated that: (i) exposure of fibrin to ribose dissolved in ethanol increases the resistance of the fibrin to proteolytic degradation; and (ii) exposure of soluble fibrinogen to ethanol resulted in formation of amorphous protein precipitate, which, in the presence of ribose, becomes water-insoluble, and its resistance to proteolytic degradation is increased.

Thus, the present inventors have successfully practiced novel processes of preparing novel crosslinked matrices of fibrin and fibrinogen.

As demonstrated in the Examples section below, the fibrin matrices and the fibrinogen matrices described herein provide a solution to the problem of fibrinolysis, as they may be prepared so as to be resistant to proteolytic degradation. As exemplified in the Examples section that follows, the degree of resistance to proteolysis can be conveniently modulated by adjusting the concentration of various reagents used to prepare the cross-linked proteins. Moreover, due to the biocompatibility of the reagent used for crosslinking these proteins, there is no danger of toxicity.

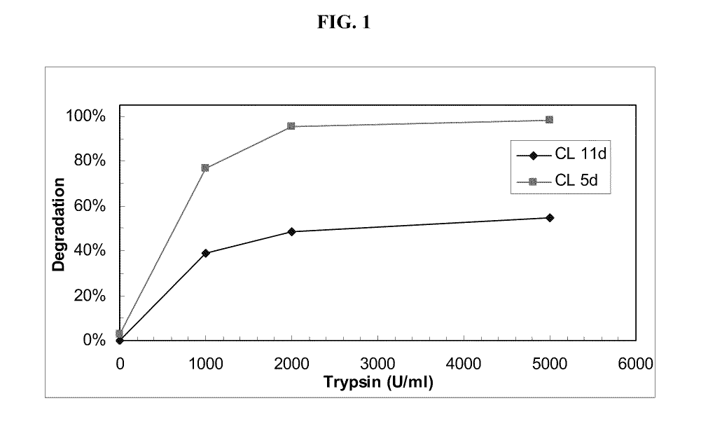

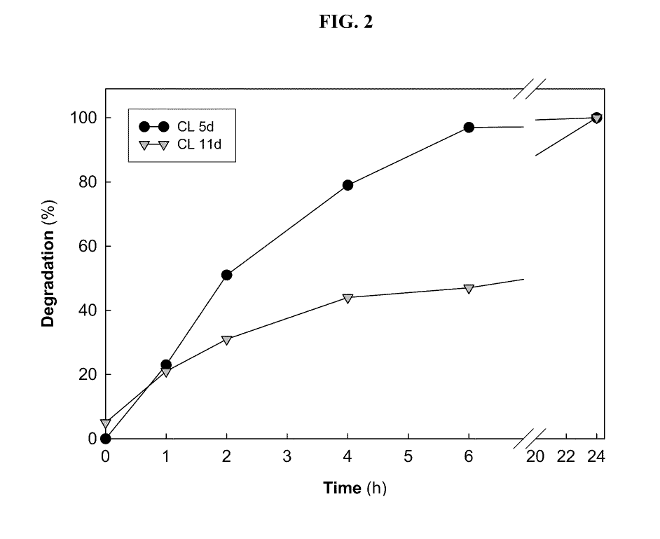

FIGS. 1 and 2 show that resistance of injectable crosslinked fibrinogen to proteolysis depends on the time during which the fibrinogen is incubated with a crosslinking solution comprising ethanol and ribose.

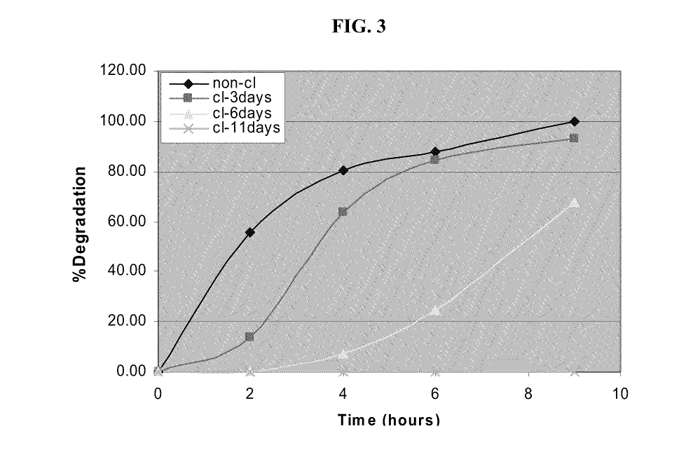

FIG. 3 shows that crosslinked fibrin (in the form of a membrane) is more resistant to proteolysis than non-crosslinked fibrin (in the form of a membrane), and that the resistance of the crosslinked fibrin to proteolysis depends on the time during which the fibrinogen is incubated with a crosslinking solution comprising ethanol and ribose.

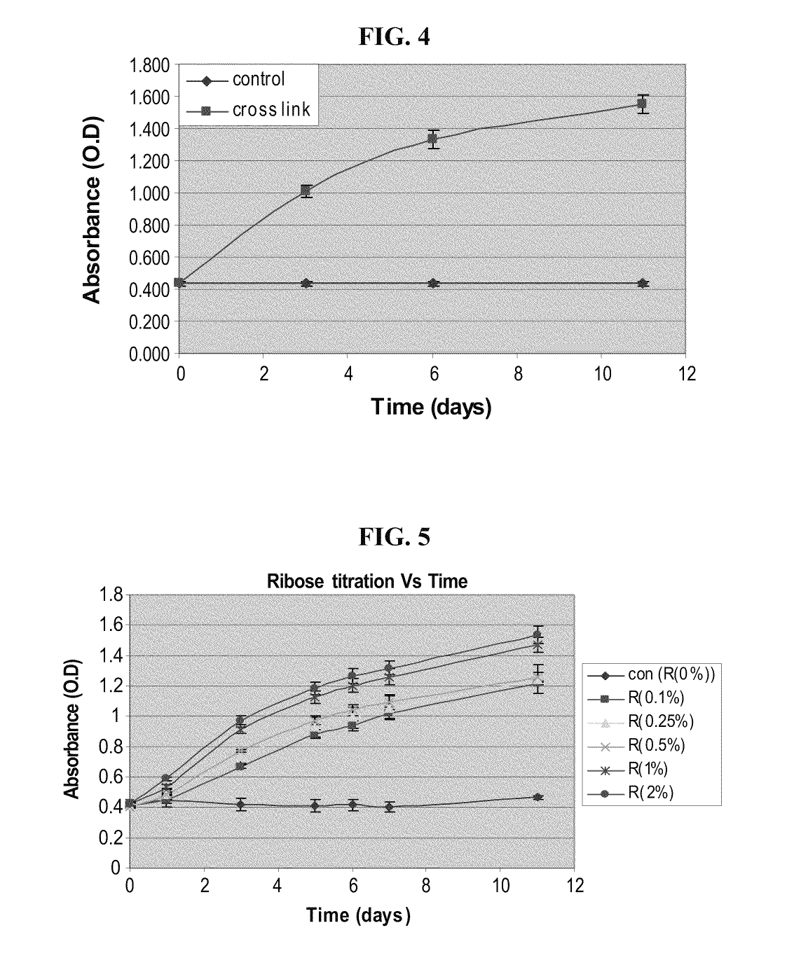

FIG. 4 shows that the crosslinking process increases the optical density of fibrin membranes.

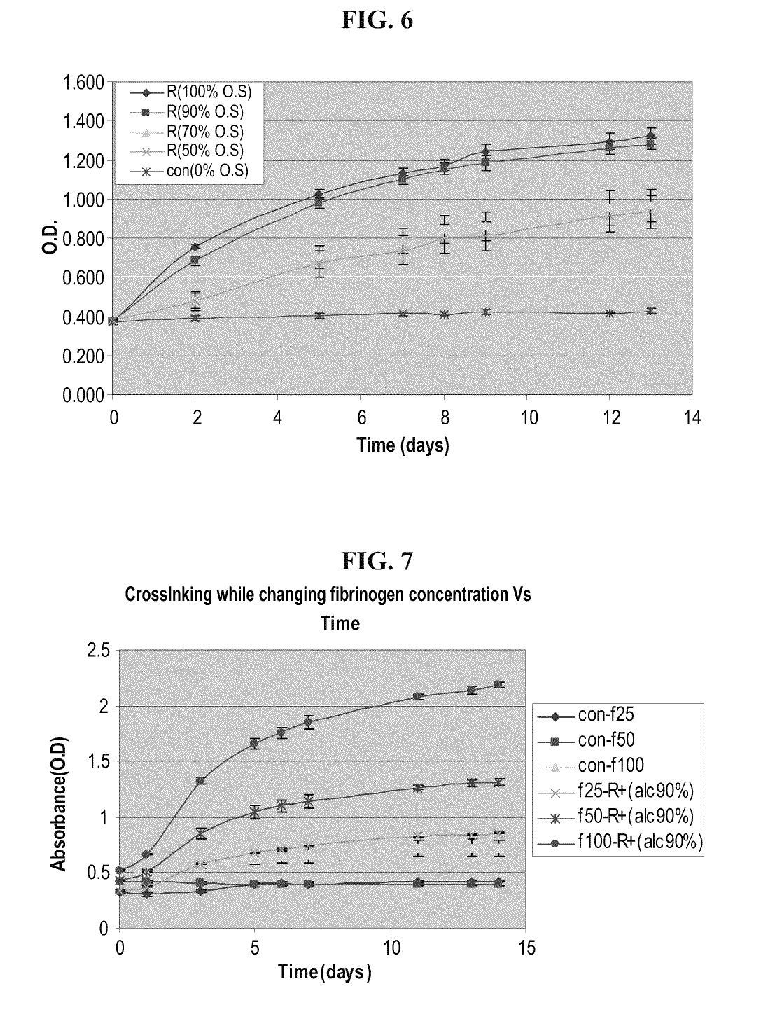

FIG. 5 shows the dependence of fibrin crosslinking kinetics in fibrin membranes on ribose concentration. FIG. 6 shows the dependence of fibrin crosslinking kinetics in fibrin membranes on organic solvent concentration. FIG. 7 shows the dependence of fibrin crosslinking kinetics in fibrin membranes on protein density of the fibrin matrix.

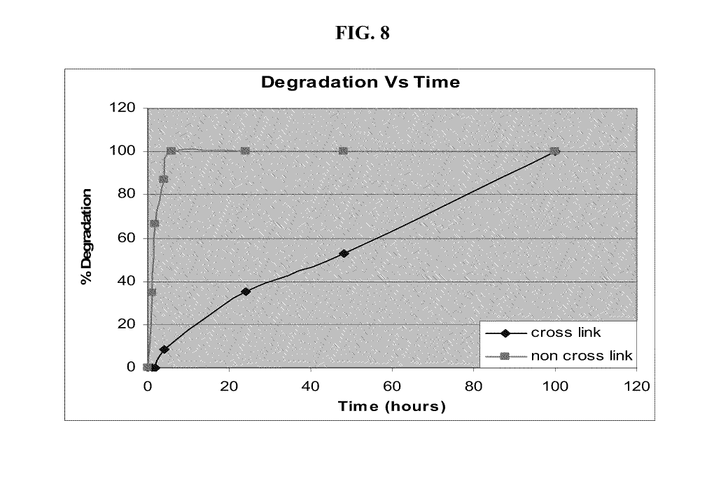

FIG. 8 shows that a crosslinked fibrin membrane is more resistant to proteolysis than is a non-crosslinked fibrin membrane.

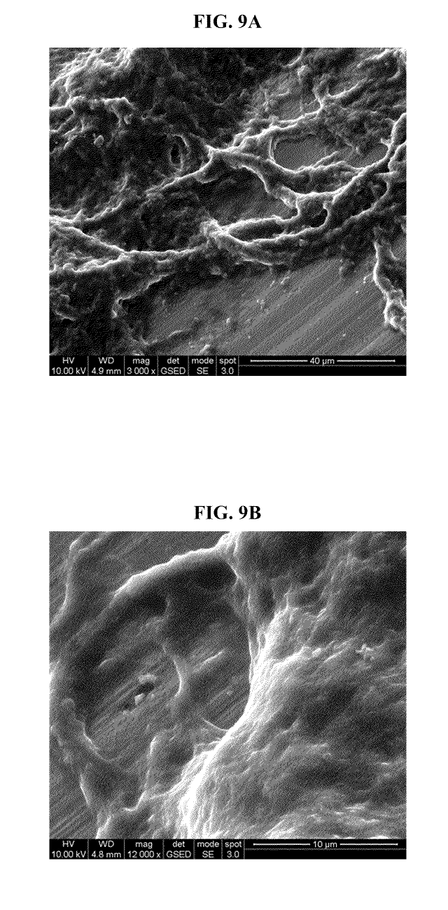

FIGS. 9A and 9B show that injectable crosslinked fibrin has a fibrillar structure.

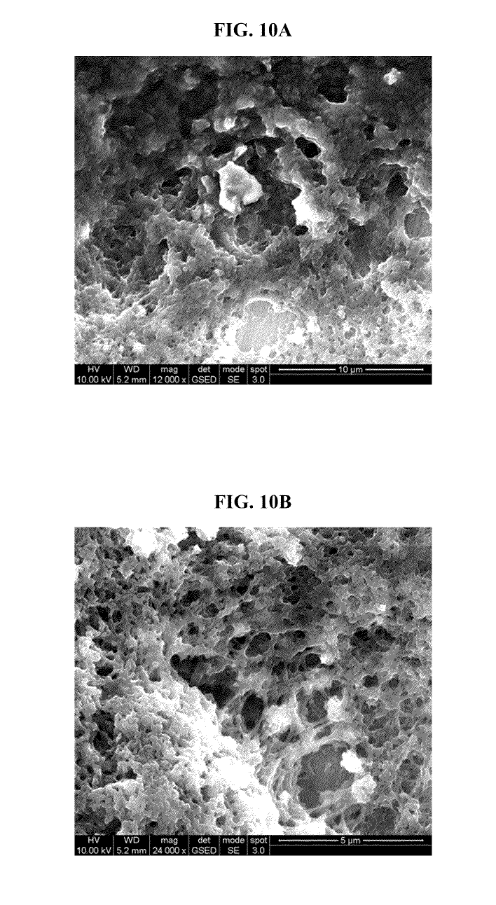

FIGS. 10A and 10B show that injectable crosslinked fibrinogen has a porous and amorphous structure.

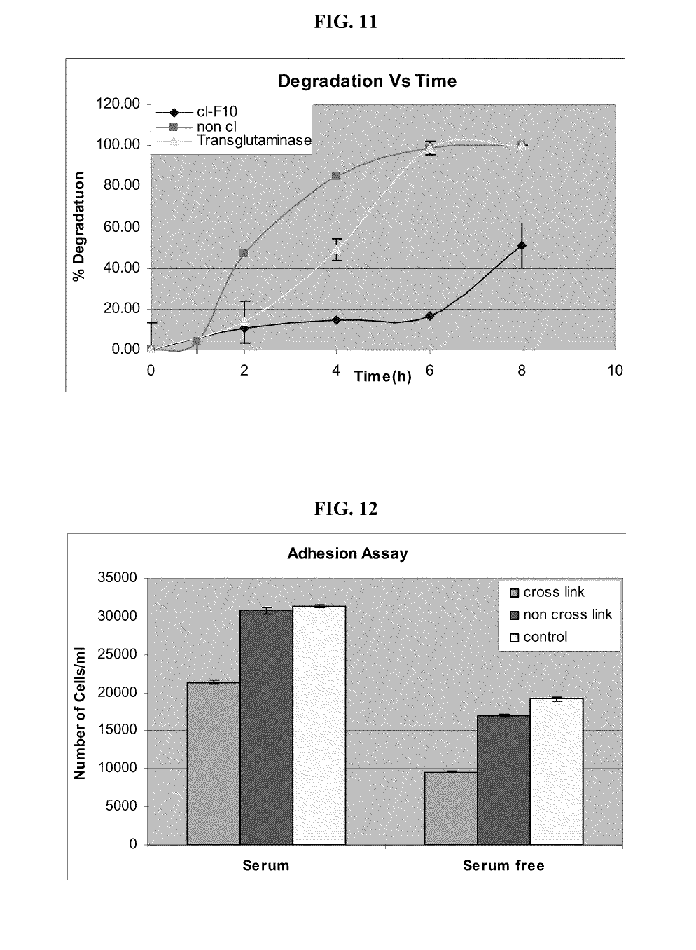

FIG. 11 shows that a fibrin matrix crosslinked according to embodiments of the present invention is more resistant to proteolysis than is a fibrin matrix crosslinked by transglutaminase.

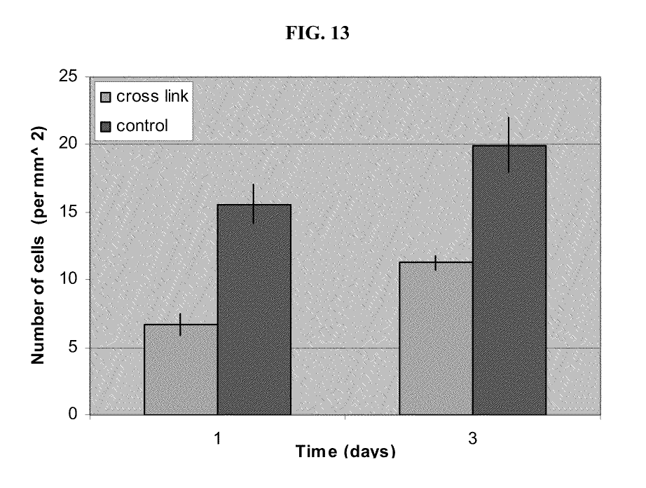

FIGS. 12 and 13 show the degree of cell adhesion and cell proliferation on crosslinked fibrin membrane surfaces.

Additional results are presented in Tables 1-3. Table 1 shows that 70% ethanol provided a greater resistance of crosslinked fibrinogen to degradation than did other concentrations of ethanol. Table 2 shows that the degradation rate for crosslinked fibrinogen is similar the degradation rate for crosslinked fibrin. Table 3 shows concentrations of fibrin and fibrinogen before and after the crosslinking process described herein.

The fibrin and fibrinogen matrices described herein can be of various longevity, and can be in the form of an injectable matrix or a non-injectable matrix, as is further detailed hereinbelow, to be used in regenerative medicine, tissue engineering and as a filler for tissue augmentation.

Hence, according to an aspect of embodiments of the present invention, there is provided a process for producing a composition-of-matter which comprises a protein being crosslinked with at least one reducing sugar. The process, according to embodiments of the invention, is effected by reacting the protein with the reducing sugar(s) in a crosslinking solution which comprises the reducing sugar and a polar solvent.

In some embodiments, the protein in fibrinogen.

According to some embodiments of the present invention, fibrinogen is crosslinked by being reacted with the reducing sugar(s) in a crosslinking solution which comprises the reducing sugar and a polar solvent.

As used herein, the terms “crosslinked” and “crosslinking” (as well as variations thereof) refer to a moiety which is bound to at least two other moieties, thereby linking those moieties to one another by acting as a bridge therebetween.

The term “matrix” is used herein interchangeably with the term “composition-of-matter”, and defines a 3-dimentional structure that is formed upon exposing a protein as described herein to crosslinking, as described herein. The matrix and composition-of-matter described herein differ in their primary, secondary, tertiary and quaternary structures from the protein used in their formation.

In the context of embodiments of the present invention, the reducing sugar crosslinks a protein (e.g., fibrinogen) by binding to at least two sites on a protein. Crosslinking may comprise both crosslinking two or more sites on a single protein molecule (intramolecular crosslinking) and/or crosslinking between two or more protein molecules (intermolecular crosslinking).

Crosslinking between a plurality of fibrinogen molecules (intermolecular crosslinking) converts the free, water-soluble fibrinogen molecules into a polymeric, water-insoluble composition-of-matter.

Optionally, crosslinking is also effected between two or more sites on the same fibrinogen molecule (intramolecular).

Crosslinking between two or more sites on a single protein may have significant effects on the properties of the protein, for example, inhibition of proteolysis.

Without being bound by any particular theory, it is believed that a reducing sugar crosslinks the protein by forming covalent bonds with amine groups of the protein. It may be suggested that covalent bonds are formed between aldehyde groups of the sugar and free amine groups, so as to form a Schiff base (an imine bond).

As used herein, a “reducing sugar” is a sugar which comprises an aldehyde group or ketone group (i.e., —C(═O)—R, wherein R is a hydrogen (for aldehyde) or an alkyl, alkenyl, cycloalkyl, or any other hydrocarbon moiety). Sugars which in aqueous solution are in equilibrium between a form comprising an aldehyde or ketone group and a form which does not comprise such a group (e.g., hemiacetal or hemiketal), are encompassed by the phrase “reducing sugar”.

In exemplary embodiments of the invention, the reducing sugar is a monosaccharide.

The term “monosaccharide”, as used herein and is well known in the art, refers to a simple form of a sugar that consists of a single saccharide unit which cannot be further decomposed to smaller saccharide building blocks or moieties. Common examples of monosaccharides which are also reducing sugars include glucose (dextrose), fructose, galactose, mannose, and ribose. Monosaccharide reducing sugars can be classified according to the number of carbon atoms of the carbohydrate, i.e., trioses, having 3 carbon atoms such as glyceraldehyde and dihydroxyacetone; tetroses, having 4 carbon atoms such as erythrose, threose and erythrulose; pentoses, having 5 carbon atoms such as arabinose, lyxose, ribose, xylose, ribulose and xylulose; hexoses, having 6 carbon atoms such as allose, altrose, galactose, glucose, gulose, idose, mannose, talose, fructose, psicose, sorbose and tagatose; heptoses, having 7 carbon atoms such as mannoheptulose, sedoheptulose; octoses, having 8 carbon atoms such as 2-keto-3-deoxy-manno-octonate; nonoses, having 9 carbon atoms such as sialose; and decoses, having 10 carbon atoms.

According to some embodiments, the monosaccharide sugar has the following formula:

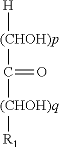

wherein R₁ is H or lower alkyl or alkylene; and p and q are each independently an integer between 0-8, whereas the sum of p and q is in a range of 2 to 8.

The above monosaccharides encompass both D- and L-monosaccharides.

According to exemplary embodiments, the reducing sugar is a pentose. Ribose (e.g., D-ribose) is an exemplary pentose.

Optionally, the reducing sugar is a disaccharide (e.g., maltose, lactose, lactulose, cellobiose, gentiobiose, melibiose, turanose), or a trisaccharide (e.g., maltotriose), i.e., a sugar comprising two or three saccharide units, respectively, and optionally an oligosaccharide, i.e., a sugar 4-10 linked saccharide units.

Alternatively, the reducing sugar can be a derivative of the above-mentioned monosaccharide, disaccharide, trisaccharide of oligosaccharide, in which a saccharide unit comprises one or more substituents other than hydroxyls. Such derivatives can be, but are not limited to, ethers, esters, amides, acids, phosphates and amines. Amine derivatives include, for example, glucosamine, galactosamine, fructosamine and mannosamine. Amide derivatives include, for example, N-acetylated amine derivatives of sugars (e.g., N-acetylglucosamine, N-acetylgalactosamine).

As used herein, the phrase “polar solvent” refers to solvents other than water (e.g., organic solvents) which are miscible with water. In exemplary embodiments, the reducing sugar(s) is soluble in the polar solvent.

The polar organic solvent can be protic (comprising releasable protons) or aprotic (devoid of releasable protons). In some embodiments, the polar solvent is an alcohol (e.g., methanol, ethanol, propanol, butanol). Ethanol is an exemplary polar solvent.

In some embodiments, the polar solvent is a pharmaceutically acceptable solvent, such as, for example, a pharmaceutically acceptable alcohol. Representative examples include, but are not limited to, ethanol, glycerol, DMSO, N,N-dimethylacetamide, propylene glycol and isopropyl alcohol.

According to some embodiments, the crosslinking solution consists essentially of the polar solvent and the reducing sugar(s).

The phrase “consisting essentially of” means that the solution may include small amounts (e.g., less than 5% by weight, less than 2% by weight, less than 1% by weight, or less than 0.5% by weight) of additional ingredients, but only if the additional ingredients do not materially alter the basic characteristics of the solution.

Alternatively, the crosslinking solution comprises additional ingredients such as water, buffering salts, etc. In exemplary embodiments, the crosslinking solution comprises phosphate buffered solution.

As mentioned hereinabove, fibrinogen is an example of a soluble protein. Protein molecules which are in solution are more difficult to crosslink, due to the lack of proximity between protein molecules. Accordingly, there are no reports in the art of crosslinking soluble (molecular) proteins.

As noted hereinabove, the present inventors have successfully practiced a process of crosslinking the water soluble fibrinogen, by performing the process in a crosslinking solution in which fibrinogen is insoluble, such that a fibrinogen precipitate is formed, so as to obtain an amorphous fibrinogen, and the amorphous fibrinogen is thereafter cross-linked by the reducing sugar.

Hence, according to some embodiments, the process further comprises precipitating fibrinogen before being crosslinked. Optionally, a polar solvent (e.g., ethanol) in which fibrinogen is insoluble is selected for inclusion in the crosslinking solution described herein, and the fibrinogen is precipitated in a solution comprising the polar solvent.

According to exemplary embodiments, the fibrinogen is precipitated in the crosslinking solution, thereby efficiently accomplishing both precipitation and crosslinking with a single crosslinking solution.

In some embodiments, the crosslinking solution comprises at least 5% (by volume) polar solvent, optionally at least 10%, at least 20%, at least 30%, at least 40%, and optionally at least 50% polar solvent, all by volume.

In some embodiments the crosslinking solution comprises from 60% to 100% by volume polar solvent. In some embodiments the crosslinking solution comprises from 60% to 80% by volume polar solvent.

As exemplified in the Examples section below, a crosslinking solution comprising 70% polar solvent was shown to be optimal for crosslinking fibrinogen.

Hence, according to some embodiments, a concentration of the polar solvent (e.g., ethanol) is about 70% (e.g., between 65% and 75%) per volume of the crosslinking solution.

According to some embodiments, a concentration of reducing sugar in the crosslinking solution is in a range of 0.1% to 6%, optionally in a range of 0.5% to 4%, and optionally in a range of 1% to 2%.

According to some embodiments, the fibrinogen is incubated in the crosslinking solution for a period of time in a range of 1 day to 20 days.

As exemplified in the Examples section below, the degree of crosslinking (e.g., inhibition of proteolytic degradation by crosslinking) is affected by the concentration of reducing sugar in the crosslinking solution, the concentration of polar solvent, and by the crosslinking time (i.e., time of incubation in the crosslinking solution), thereby allowing one of skill in the art to obtain a desired level of crosslinking by selecting a suitable sugar concentration, polar solvent concentration and/or crosslinking time.

Hence, according to some embodiments, the concentration of reducing sugar and/or the crosslinking time is selected according to the desired properties of the composition-of-matter. As exemplified in the Examples section, a person of skill in the art may readily assay the relevant property (e.g., resistance to proteolytic degradation, mechanical strength, protein density) in compositions-of-matter prepared using various sugar concentrations, polar solvent concentrations and/or crosslinking times, thereby allowing the skilled person to determine which sugar concentration, polar solvent concentration and/or crosslinking time will provide a composition-of-matter with the desired property.

Fibrinogen is optionally added to a crosslinking solution at a concentration in a range of 0.5 mg/ml to 50 mg/ml, optionally in a range of 1 mg/ml to 25 mg/ml, optionally in a range of 2.5 mg/ml to 20 mg/ml, optionally in a range of 1 mg/ml to 10 mg/ml, optionally in a range of 2.5 mg/ml to 10 mg/ml, optionally in a range of 2 mg/ml to 6 mg/ml, and optionally in a range of 5 mg/ml to 10 mg/ml.

It is to be appreciated that following precipitation and crosslinking, the concentration of fibrinogen in the prepared composition-of-matter may differ from the initial concentration, as exemplified in the Examples section that follows.

According to some embodiments, the crosslinked composition-of-matter is centrifuged. Centrifugation may also increase the concentration of fibrinogen by making the crosslinked fibrinogen matrix denser.

Thus, according to some embodiments, a final concentration of fibrinogen in the composition-of-matter is in a range of 1 mg/ml to 100 mg/ml, optionally in a range of 1 mg/ml to 50 mg/ml, optionally in a range of 2 mg/ml to 50 mg/ml, optionally in a range of 5 mg/ml to 25 mg/ml, optionally in a range of 10 mg/ml to 25 mg/ml and optionally in a range of 15 mg/ml to 20 mg/ml.

In some embodiments, the obtained composition-of-matter, which comprises cross-linked fibrinogen, can be further subjected to drying.

Drying can be effected, for example, by critical point drying (CPD), by lyophilization, or by any other drying method that does not affect the structural and chemical properties of the final product.

As discussed hereinabove, the present inventors have also successfully practiced a process of preparing crosslinked fibrin, being crosslinked with a reducing sugar, as described herein. The present inventors have practiced a crosslinking process that involves contacting fibrin with a crosslinking solution that comprises a reducing sugar and a polar organic solvent, and have identified the process parameters that result in crosslinked fibrin that have desired characteristics.

Hence, according to embodiments of another aspect of the present invention, there is provided a process for producing a composition-of-matter which comprises fibrin being crosslinked with at least one reducing sugar. The process, according to these embodiments, is effected by reacting fibrin with at least one reducing sugar in a crosslinking solution which comprises the reducing sugar, as described herein and a polar solvent (as described herein, e.g., ethanol).

It is to be appreciated that fibrin per se exists as a polymeric, insoluble matrix comprising fibrils, each fibril comprising many fibrin molecules. Although fibrin is crosslinked in vivo by Factor XIII, it is to be understood that the term “fibrin” herein refers to fibrin which has not been crosslinked by Factor XIII (e.g., fibrin produced in vitro), unless indicated otherwise. Thus, the term “fibrin”, when used per se, describes non-crosslinked fibrin, typically in a form of fibrillar insoluble structure (e.g., in a form of a non-injectable composition).

Crosslinking fibrin may comprise both crosslinking two or more sites on a single fibrin fiber and/or crosslinking between two or more fibrin fibers in a single fibril and/or crosslinking between two or more fibrils.

Crosslinking of fibrin according to embodiments of the present invention alters the properties of the fibrin matrix, for example, by inhibiting proteolysis and/or strengthening mechanical properties.

The reducing sugar, crosslinking solution and polar solvent are as described herein.

In some embodiments, the concentration of the reducing sugar is in a range of 0.1% to 6%.

Herein throughout, concentrations of a reducing sugar in a solution are calculated as weight per volume solution.

According to some embodiments, a concentration of the reducing sugar is in a range of 0.1% to 4%, optionally in a range of 0.1% to 2%, optionally in a range of 0.5% to 2%, and optionally in a range of 1% to 2%. According to exemplary embodiments, the concentration is about 1%.

Thus, as exemplified in the Examples, a crosslinking solution with a concentration of reducing sugar in a range of about 0.1% to about 0.5% provides a moderate degree of crosslinking of fibrin, whereas a concentration in a range of about 1% to about 2% provides a high degree of crosslinking.

According to some embodiments, the fibrin is incubated in the crosslinking solution for a period of time in a range of 1 day to 20 days. As exemplified in the Examples, longer incubation times (e.g., at least 10 days) result in a higher degree of crosslinking.

According to exemplary embodiments, a concentration of polar solvent is at least 50% per volume of the crosslinking solution, optionally at least 80%, and optionally at least 90%. As exemplified below, a crosslinking solution with a concentration of organic solvent in a range of about 50% to about 70% provides a moderate degree of crosslinking of fibrin, whereas a concentration in a range of about 90% to about 100% provides a high degree of crosslinking.

In many cases, it will be advantageous to prepare a crosslinked fibrin matrix described herein from the precursor of fibrin, i.e., fibrinogen. Such a procedure provides fresh fibrin for crosslinking, and also allows one to select fibrin with whatever specific parameters (e.g., protein density) may be desired for crosslinking.

Hence, according to optional embodiments, the process further comprises reacting fibrinogen with thrombin so as to obtain fibrin, prior to reacting the fibrin with the reducing sugar(s) as described hereinabove.

As exemplified in the Examples section, the properties of crosslinked fibrin may be modified by using different concentrations of fibrinogen when preparing the fibrin.

Optionally, fibrin is prepared using a concentration of fibrinogen in a range of from 1 mg/ml to 300 mg/ml, optionally from 3 mg/ml to 100 mg/ml, and optionally, from 25 mg/ml to 100 mg/ml.

As exemplified in the Examples, an initial fibrinogen concentration of up to about 25 mg/ml results in a relatively low degree of crosslinking, an initial fibrinogen concentration of about 50 mg/ml results in an intermediate degree of crosslinking, and an initial fibrinogen concentration of at least about 100 mg/ml results in a high degree of crosslinking.

As further exemplified in the Examples, the concentration of fibrin in the final composition-of-matter (i.e., following crosslinking) may be higher than the initial concentration of fibrinogen.

In some embodiments, the concentration of fibrin in the crosslinked composition-of-matter is in a range of about 3 mg/ml to about 200 mg/ml, optionally in a range of about 10 mg/ml to about 150 mg/ml, and optionally in a range of 25 mg/ml to 100 mg/ml.

As discussed hereinabove and exemplified in the Examples section below, the degree of crosslinking is affected by various parameters of the crosslinking process.

Consequently a person of skill in the art may readily select a desired property of a fibrin matrix, for example, by assaying a relevant property (e.g., resistance to proteolytic degradation, mechanical strength, protein density) or by measuring optical density of compositions-of-matter prepared using various sugar concentrations, polar solvent concentrations, initial fibrinogen concentrations, and/or crosslinking times.

Each of the processes described herein may be designed so as to produce an injectable form of a composition-of-matter or a non-injectable form of a composition-of-matter.

As used herein, the phrase “injectable form” refers to a composition-of-matter (e.g., comprising crosslinked fibrinogen and/or crosslinked fibrin) in the form of particles small enough to allow for injection into a human subject (e.g., injection via a syringe and needle), as well as being of a suitable purity and non-toxicity for injection into a subject. The composition of matter should be homogeneous and have the rheological properties for passing smoothly while injected through needles of various internal diameters (14-32 gauge).

Optionally, the injectable form of the composition-of-matter is mixed with a suitable carrier.

The injectable form may be a liquid form, a paste, an emulsion, a dispersion or a particulated solid form. The solid particles therein are capable of passing through an injection device (e.g., a 14-32 gauge needle)

As exemplified in the Examples section, an injectable form of a composition-of-matter described herein may optionally be prepared by subjecting the composition-of-matter to particulation.

As used herein, the term “particulation” encompasses converting to a particulate form and/or reducing the size of particles. Particulation may be, for example, by breaking, crushing, grinding, pressuring through a mesh and/or a narrow needle (e.g., a 21 G needle and/or whatever gauge needle is desired to be used for injection), and/or homogenization (e.g., a Dounce homogenizer, sonification

An injectable form can also be an inherent product of the process described herein, as in the case of fibrinogen. Thus, in some embodiments, subjecting fibrinogen to a crosslinking solution results in a composition-of-matter in the form of fine particles (e.g., in the form of a paste) which are injectable, and therefore do not require any further processing to be in an injectable form.

A “non-injectable” composition-of-matter may be designed in various shapes and sizes, for example, as a membrane or a pre-determined 3-dimensional shape (e.g., by crosslinking in a mold with the desired shape, or by first forming a non-crosslinked fibrin matrix with the desired 3-dimensional shape).

Each of the processes described herein is optionally performed at a temperature ranging from 5° C. to 41° C., optionally from 20° C. to 38° C. 37° C. is an exemplary temperature for crosslinking.

As discussed hereinabove, the processes described herein provide fibrinogen matrices and fibrin matrices with improved properties (e.g., resistance to proteolysis), wherein the properties of the matrices can be predetermined by adjusting various parameters (e.g., time of incubation in the crosslinking solution and/or composition of the crosslinking solution).

According to an aspect of embodiments of the present invention there is provided cross-linked fibrinogen. As discussed hereinabove, the present inventors were successful in performing a task difficult to achieve—crosslinking of soluble proteins.

In some embodiments, there is provided a composition-of-matter which comprises cross-linked fibrinogen.

In some embodiments, the composition-of-matter comprises fibrinogen crosslinked with a reducing sugar, as described herein.

Optionally, the composition-of-matter is obtainable by a process described herein.

According to exemplary embodiments, the composition-of-matter is characterized by a structure (e.g., a structure as viewed by electron scanning microscopy) comprising an aggregation of microparticles.

As used herein, the term “microparticles” refers to particles having a diameter in a range of 100 microns or less, optionally in a range of 0.01 microns to 10 microns (e.g., 0.1 microns to 2 microns). Microparticles are distinct from fibrils in that fibrils by nature are typically of highly variable length and diameter, whereas microparticles are spheroid or at least close enough to being spheroid so as to be characterized by a diameter thereof.

Optionally, the structure is amorphous (e.g., as viewed by electron scanning microscopy).

According to exemplary embodiments, the composition-of-matter comprising crosslinked fibrinogen is injectable.

According to exemplary embodiments, the composition-of-matter comprising crosslinked fibrinogen has an appearance of a paste (e.g., a non-opaque dispersion in which individual particles are not visible to the naked eye).

According to some embodiments, the composition-of-matter comprising crosslinked fibrinogen is in a non-injectable form (e.g., a membrane, a large matrix).

According to an aspect of embodiments of the present invention there is provided cross-linked fibrin.

In some embodiments, there is provided a composition-of-matter which comprises cross-linked fibrin.

In some embodiments, the composition-of-matter comprises fibrin crosslinked with a reducing sugar, as described herein.

Optionally, the composition-of-matter is obtainable by a process described herein.

According to exemplary embodiments, the composition-of-matter comprising crosslinked fibrin is characterized by a fibrillar structure, such that a mesh of distinct fibrils is observable (e.g., as viewed by electron scanning microscopy).

According to optional embodiments of the present invention, the composition-of-matter comprising crosslinked fibrinogen is designed so as to have a predetermined resistance to proteolytic degradation. Resistance to proteolytic degradation may be characterized as the degradation time of the composition-of-matter when subjected to 1000 units/ml trypsin at 37° C., as exemplified herein. The predetermined resistance to proteolytic degradation is optionally selected as any degradation time in a range of 1 hour to 7 days under the aforementioned conditions.

According to exemplary embodiments, the composition-of-matter comprising crosslinked fibrin is injectable.

According to further exemplary embodiments, the composition-of-matter comprising crosslinked fibrin is in a non-injectable form (e.g., a membrane, a large matrix, an artificial clot).

According to some embodiments, the composition-of-matter comprising crosslinked fibrin exhibits a higher resistance to proteolytic degradation than a Factor XIIIa-crosslinked fibrin matrix.

Optionally, the higher resistance to proteolytic degradation is characterized as a longer degradation time when exposed to proteolytic degradation. Alternatively, resistance to proteolytic degradation is characterized as a half-life under proteolytic conditions.

Optionally, the degradation time (or half-life) of the composition-of-matter according to embodiments of the present invention is 20% longer, optionally 50% longer, and optionally 100% longer, than the corresponding degradation time (or half-life) of a Factor XIIIa-crosslinked fibrin matrix.

As used herein, the phrase “degradation time” refers to the time until substantially all of the tested substance has been degraded. A substance is herein considered to be degraded when the substance has been broken down such that no visible portion remains.

As used herein, the term “half-life” refers to the time until 50% of the tested substance has been degraded.

To compare resistance of the different matrices, a composition-of-matter according to embodiments of the present invention and a Factor XIIIa-crosslinked fibrin matrix having the same dimensions and protein content, are subjected to proteolytic conditions. Optionally, the proteolytic conditions comprise placing the matrices in a solution comprising trypsin (e.g., 1000 units/ml trypsin) for several hours (e.g., 2 hours, 4 hours, 6 hours, 24 hours), or as long as is necessary to determine the relevant degradation time or half-life.

As exemplified in the Examples section, crosslinking of fibrin according to some embodiments of the present invention results in an increase in optical density.

Thus, in some embodiments, the composition-of-matter described herein has an optical density at least 20% higher, optionally at least 50% higher, optionally at least 100% higher, and optionally 200% higher, than the optical density of a non-crosslinked fibrin matrix having the same dimensions and fibrin content as the composition-of-matter.

Measurement of the optical densities of the crosslinked and non-crosslinked fibrin may be performed according to standard spectroscopic procedures used in the art. Crosslinked and non-crosslinked samples may be measured suspended in a liquid, wherein the liquids in the different samples are identical, or at least have substantially the same optical properties (e.g., absorption, refractive index).

As used herein, the “optical density” refers to −1 multiplied by the logarithm of the fraction of light which passes through a sample. It is to be appreciated that the optical density of a sample represents both loss of light due to absorption and loss of light due to scattering.

According to optional embodiments of the present invention, the composition-of-matter is designed so as to have a predetermined resistance to proteolytic degradation. Resistance to proteolytic degradation may be characterized as the degradation time of the composition-of-matter when subjected to 1000 units/ml trypsin at 37° C., as exemplified herein. The predetermined resistance to proteolytic degradation is optionally selected as any degradation time in a range of 1 hour to 7 days under the aforementioned conditions.

According to some embodiments, the composition-of-matter absorbs liquid (e.g., aqueous solution). Optionally, a gel (e.g., hydrogel) is formed from the composition-of-matter by absorption of the liquid.

Any of the compositions-of-matter described herein (e.g., a hydrogel) may optionally further comprise a pharmaceutically active agent, being contained within the composition-of-matter or on a surface of the composition-of-matter.

The pharmaceutically active agent is optionally covalently attached to the crosslinked protein (i.e., fibrinogen or fibrin) matrix (e.g., covalently linked to the protein and/or to the sugar. The agent may be covalently attached to the matrix after the matrix has been prepared and/or covalently attached to the ingredients (e.g., protein and/or reducing sugar) which are then used to prepare the crosslinked matrix.

Alternatively or additionally, the agent is absorbed by the crosslinked protein matrix. Absorption may be obtained, for example, by contacting a crosslinked fibrinogen or fibrin matrix or a non-crosslinked fibrin matrix with a solution containing the agent (e.g., by dipping, soaking, washing), or by adding the agent prior to crosslinking (e.g., inclusion of the agent in the crosslinking solution, inclusion of the agent in a solution of fibrinogen and thrombin).

Alternatively or additionally, the agent may be entrapped in the protein matrix without being bound to the matrix, for example, by adding the agent prior to crosslinking (e.g., inclusion of the agent in the crosslinking solution, inclusion of the agent in a solution of fibrinogen and thrombin).

Non-limiting examples of pharmaceutically active agents which may be applied include an agent for promoting tissue regeneration, an agent for promoting healing, and a drug to be delivered (e.g., when the composition-of-matter is used as a drug delivery system).

According to some embodiments, the pharmaceutically active agent is a therapeutically active agent and/or a labeling agent. Exemplary therapeutically active agents that are suitable for use in the context of some embodiments of the invention include, but are not limited to, a stem cell, a growth factor, a morphogenetic protein, a cell, a cytokine, a hormone, a medicament, a mineral, a plasmid with therapeutic potential, and a combination of thereof.

Examples of suitable growth factors include an epidermal growth factor, a nerve growth factor, a vascular endothelial growth factor, an insulin-like growth factor (e.g., insulin-like growth factor-1), a transforming growth factor (e.g., transforming growth factor-β), and a fibroblast growth factor (e.g., basic fibroblast growth factor).

Examples of suitable bone morphogenic proteins (BMPs) include BMP-2, BMP-7, cartilage-inducing factor-A, cartilage-inducing factor-B, osteoid-inducing factor, collagen growth factor and osteogenin.

Stem cells may be beneficial in that they may optionally secrete substances (e.g., growth factors) having a beneficial effect (e.g., promoting tissue growth and/or promoting wound healing). Alternatively or additionally, the stem cells may undergo differentiation to a desired cell type (e.g., a cell type of a tissue which is to be regenerated).

A medicament is optionally included in the composition-of-matter so as to produce a drug delivery system wherein the medicament is releases in a controlled manner. Examples of suitable medicaments include a chemotherapeutic agent and an antibiotic.

A suitable mineral is optionally a mineral conductive to bone development (e.g., a mineral comprising calcium and/or a mineral comprising phosphate).

Exemplary labeling agents that are suitable for use in the context of some embodiments of the invention include, but are not limited to, fluorescent agents, phosphorescent agents, chromophores, radioactive agents, contrast agents, metal clusters, and more.

The incorporation of a labeling agent can be onto or into the composition-of-matter as described herein, either alone, or in combination with a therapeutically active agent. Alternatively, the labeling agent can be attached to, or form a part of, the therapeutically active agent.

The incorporation of a labeling agent allows monitoring the composition-of-matter upon its incorporation in a medium, for example, upon administration of the composition-of-matter to a subject's body.

As further described herein, the compositions-of-matter described herein can be used in a variety of clinical and cosmetic applications, such as in tissue regeneration and wound healing.

Thus, in some embodiments, the composition-of-matter is identified for use in the treatment of a medical disorder or a cosmetic disorder characterized by a tissue damage.

According to another aspect of embodiments of the present invention, there is provided a use of a composition-of-matter described herein in the manufacture of a medicament for the treatment of a medical disorder or a cosmetic disorder characterized by a tissue damage.

According to another aspect of embodiments of the present invention, there is provided a method of treating a medical disorder or a cosmetic disorder characterized by a tissue damage, the method comprising administering to a subject in need thereof a therapeutically effective amount of a composition-of-matter described herein.

A “therapeutically effective amount” means an amount effective to prevent, alleviate, or ameliorate symptoms of a disorder or prolong the survival of the subject being treated.

A “medical disorder or cosmetic disorder characterized by a tissue damage” refers to a disorder, disease or condition which is caused by, or associated with, a non-functioning tissue (e.g., cancerous or pre-cancerous tissue, wounded tissue, broken tissue, fractured tissue, fibrotic tissue, or ischemic tissue); and/or tissue loss (reduced amount of functioning tissue) such as following a trauma, an injury or abnormal development (e.g., malformation, structural defect that occurs infrequently such as due to abnormal development which require tissue regeneration). In some embodiments, the tissue is a functional tissue such as a bone tissue, a cartilage tissue, a tendon tissue, ligament, a cardiac tissue, a nerve tissue, or a muscle tissue.

Examples of disorders characterized by a tissue damage include, but are not limited to, cartilage damage (articular, mandibular), bone cancer, osteoporosis, bone fracture or deficiency, primary or secondary hyperparathyroidism, osteoarthritis, periodontal disease or defect, an osteolytic bone disease, post-plastic surgery, post-orthopedic implantation, post-dental implantation, cardiac ischemia, muscle atrophy, nerve degeneration, skin burns, wrinkles, scarring, irradiation damages, incontinence consequent to muscle incompetence, gastroesophageal reflux disease, fecal and urinary incontinence, chronic heart failure and nucleus pulposus pathologies.

According to optional embodiments, the disorder is a disorder which is treatable by a procedure selected from the group consisting of tissue regeneration (e.g., enhancing growth of new tissue), wound healing (e.g., increasing a rate of healing), tissue engineering, drug delivery (e.g., releasing a drug from the composition-of-matter in a localized manner and/or at a controlled rate), and tissue augmentation (e.g., adding to existing tissue, enlarging a volume of a tissue).

Thus, according to another aspect of embodiments of the present invention, there is provided a method of performing tissue regeneration, wound healing, tissue engineering, drug delivery, and/or tissue augmentation in a subject in need thereof, the method comprising administering to the subject a composition-of-matter described herein.

A composition-of-matter described herein can be formulated for local or systemic administration.

Thus, according to another aspect of the present invention, there is provided a pharmaceutical, cosmetic or cosmeceutical composition comprising a composition-of-matter described herein and a carrier (i.e., a pharmaceutically, cosmetically or cosmeceutically acceptable carrier, in accordance with the type of composition).

The term “cosmeceutical” refers to a composition having both cosmetic and pharmaceutical effects.

Techniques for formulation and administration of pharmaceutical compositions may be found in the latest edition of “Remington's Pharmaceutical Sciences,” Mack Publishing Co., Easton, Pa., which is herein fully incorporated by reference.

Suitable routes of administration may, for example, include oral, rectal, transmucosal, especially transnasal, intestinal, or parenteral delivery, including intramuscular, subcutaneous, and intramedullary injections, as well as intrathecal, direct intraventricular, intravenous, inrtaperitoneal, intranasal, or intraocular injections.

According to some embodiments, the composition-of-matter and/or the pharmaceutical, cosmetic or cosmeceutical composition is administered in a local rather than systemic manner.

Thus, according to some embodiments, the composition-of-matter is administered to a subject by implantation (e.g., surgical implantation of the composition-of-matter directly into a tissue region (e.g., a damaged tissue) of a patient).

According to some embodiments, the composition-of-matter is administered to a subject by injection (e.g., injection via a needle directly into a tissue region of a patient). In these embodiments, a composition-of-matter in an injectable form is utilized.

Compositions of the present invention may be manufactured by processes well known in the art, e.g., by means of conventional mixing, dissolving, granulating, dragee-making, levigating, emulsifying, encapsulating, entrapping, or lyophilizing processes.

For injection, an injectable form of the composition-of-matter described hereinabove may be formulated in aqueous solutions, preferably in physiologically compatible buffers such as Hank's solution, Ringer's solution, or physiological salt buffer.

For transmucosal administration, penetrants appropriate to the barrier to be permeated are used in the formulation. Such penetrants are generally known in the art.

Pharmaceutical compositions suitable for use in the context of the present invention include compositions wherein a composition-of-matter is contained in an amount effective to achieve the intended purpose, for example an amount effective to prevent, alleviate, or ameliorate symptoms of a disorder or prolong the survival of the subject being treated.

Determination of a suitable amount of composition-of-matter to be administered is within the capabilities of the ordinary skilled artisan, and will depend, for example, on the nature of the application and the size of the area being treated.

As mentioned hereinabove, the composition-of-matter is relatively non-toxic, being comprised of non-toxic protein and sugars. However, according to some embodiments, the composition-of-matter further comprises a further agent (e.g., a therapeutically active agent), which may exhibit some toxicity.

Toxicity and therapeutic efficacy of a composition-of-matter per se (e.g., the crosslinked protein matrix) and/or a therapeutically active agent contained by the composition-of-matter can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration, and dosage can be chosen by the individual physician in view of the patient's condition. (See, e.g., Fingl, E. et al. (1975), “The Pharmacological Basis of Therapeutics,” Ch. 1, p. 1.)

According to another aspect of embodiments of the present invention, a kit is provided for generating a composition-of-matter described herein.

According to some embodiments, a kit for preparing a composition-of-matter comprising crosslinked fibrinogen is provided, the kit comprising: (i) fibrinogen; and (ii) a reducing sugar.

Optionally, the fibrinogen and reducing sugar are each packaged individually (e.g., in dry form or in solution) in the kit. Thus, each of the two ingredients is packaged in separate packaging material, in addition to the packaging material of the whole kit.

Alternatively, the fibrinogen and reducing sugar are packaged together (e.g., as a dry mixture) in the kit in the same packaging material.

In some embodiments, the kit further comprises instructions on how to combine the ingredients of the kit and/or how to combine the ingredients of the kit with an additional ingredient (e.g., a suitable polar solvent, a crosslinking solution comprising a polar solvent), in order to produce the desired composition-of-matter.

According to alternative embodiments, a kit for preparing a composition-of-matter comprising crosslinked fibrin is provided, the kit comprising: (i) fibrinogen; (ii) thrombin; and (iii) a reducing sugar.

Optionally, the fibrinogen, thrombin and reducing sugar are each packaged individually (e.g., in dry form or in solution) in the kit. Thus, each of the three ingredients is packaged in separate packaging material, in addition to the packaging material of the whole kit.

Alternatively, the fibrinogen, thrombin and reducing sugar are packaged together (e.g., as a dry mixture) in the kit in the same packaging material.

Optionally, the kit further comprises a polar solvent. The polar solvent may be in a pure form or in a solution with another liquid (e.g., water, aqueous buffer). Optionally, the polar solvent (or the solution comprising the polar solvent) is packaged individually, apart from the fibrinogen and reducing sugar (or fibrinogen, thrombin and reducing sugar). Alternatively, the polar solvent is packaged in combination with the reducing sugar, for example, as a ready-for-use crosslinking solution described herein.

Compositions described herein, as well as the contents of an above-mentioned kits, may, if desired, be presented in a pack or dispenser device, such as an FDA-approved kit, which may contain one or more unit dosage forms containing the composition-of-matter or ingredients (e.g., fibrinogen, reducing sugar) and/or reagents (e.g., polar solvent, thrombin) for preparing the composition-of-matter. The pack may, for example, comprise metal or plastic foil, such as a blister pack. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising a preparation of the invention formulated in a pharmaceutically acceptable carrier may also be prepared, placed in an appropriate container, and labeled for use for an indicated application and/or for treatment of an indicated condition, as further detailed above.

It will be appreciated that compositions-of-matter of embodiments of the present invention may be attached to or included in medical devices, such as for promoting wound healing following implantation or promoting cell settling on the implant.

Hence, according to a further aspect of embodiments of the present invention, there is provided a medical device composed of, or comprising, a composition-of-matter described herein.

Examples of medical devices which can be used in accordance with the present invention include, but are not limited to, intracorporeal or extracorporeal devices (e.g., catheters), temporary or permanent implants, stents, vascular grafts, anastomotic devices, prosthetic device, pacemaker, aneurysm repair devices, embolic devices, and implantable devices (e.g., orthopedic (e.g., an artificial joint) and orthodental implants), aneurysm repair devices and the like. Optionally, the device is in a form of a membrane. Other devices which can be used in accordance with the present invention are described in U.S. Pat. Appl. No. 20050038498.

As used herein the term “about” refers to ±10%.

The terms “comprises”, “comprising”, “includes”, “including”, “having” and their conjugates mean “including but not limited to”. This term encompasses the terms “consisting of” and “consisting essentially of”.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate some embodiments of the invention in a non-limiting fashion.

### Materials and Methods

Materials:
Fibrinogen from bovine plasma (Fraction I, type I-S) was obtained from Sigma;
Thrombin (human or bovine) was obtained from Sigma;
Transglutaminase (factor XIIIa) was obtained from Sigma;
Trypsin was were obtained from Sigma;
Dulbecco's phosphate buffered saline (PBS) with Ca⁺⁺ and Mg⁺⁺ was obtained from Biological Industries (Israel);
Ethanol (absolute) was obtained from Merck;
Hydrochloric acid was obtained from Merck;
Sodium hydroxide was obtained from Merck;
D-ribose was obtained from Sigma, and dissolved at a concentration of 40% to form a stock solution.
Alpha Minimum Essential Medium and Fetal calf serum was obtained were obtained from Biological Industries, Israel;

Preparation of Crosslinked Fibrinogen (CL-Fibrinogen):

A 10 ml solution of fibrinogen in PBS (phosphate buffered saline), ribose and ethanol was prepared. Addition of at least approximately 50% (v/v) ethanol resulted in precipitation of the fibrinogen. Except when indicated otherwise, 50 mg fibrinogen was dissolved in 2.75 ml PBS, and 0.25 ml of 40% ribose solution and 7 ml of ethanol were added.

The solution was then collected into a 10 ml syringe and passed through a 21 G needle or a needle of smaller size, in order to obtain an injectable material. The resulting material was placed in a sterile vial and incubated at 37° C. for the indicated time period. At the end of incubation, the mixture was transferred into a tube and centrifuged at a force of 1200 g (2500 rotations per minute, 10 minutes), and the pellet was collected. The cross-linked (CL)-fibrinogen pellet was washed twice by re-suspending the pellet in PBS followed by centrifugation.

When ribose was not included in the fibrinogen solution, the obtained pellet dissolved when resuspended in PBS, thereby confirming that the insoluble pellets consist primarily of CL-fibrinogen.

Washed pellets were subjected to a protein concentration assay in order to determine the yield, which was defined as the percentage of the original fibrinogen recovered in the CL-fibrinogen.

Preparation of Crosslinked Fibrin (CL-Fibrin):

A 5 ml solution of 20 mg/ml fibrinogen in PBS was mixed in a 10 ml syringe with a 5 ml solution of 2000 units/ml thrombin in PBS, unless indicated otherwise. A fibrin gel appeared, and after incubation for 1 hour at 37° C., the gel was dispersed by being passed through a 21 G needle. The fibrin was then collected by centrifugation at a force of 1200 g for 10 minutes. The obtained pellet was dispersed at a fibrin concentration of 10 mg/ml in 70% ethanol with 1% ribose, and incubated without movement at 37° C. for the indicated time period. At the end of incubation, the mixture was transferred into a tube and centrifuged at a force of 1200 g (2500 rotations per minute) for 10 minutes, and the pellet was collected. The CL-fibrinogen pellet was washed twice by re-suspending the pellet in PBS followed by centrifugation.

Washed pellets were subjected to a protein concentration assay in order to determine the yield, which was defined as the percentage of the original fibrinogen recovered in the CL-fibrin.

Trypsin Degradation Assay:

Trypsin was dissolved at a concentration of 2000 units/ml in a solution of 50:1 (v/v) PBS: HCl solution.

A sample (100-200 μl) of the tested protein (CL-fibrinogen or CL-fibrin) was placed into a pre-weighed tube and centrifuged (3500 rotations per minute, 5 minutes), and the supernatant was then discarded. The tube was then weighed in order to determine the net weight of the pellet (typically 50-100 mg). 500 μl of PBS:HCl (50:1, v/v) was added to the crosslinked protein pellet, and the mixture was vortexed until a homogeneous dispersion of material was achieved.

500 μl of the trypsin solution was added to the samples, and the samples were then incubated at 37° C. for up to 24 hours. At a predetermined time, a sample of 300 μl was taken and centrifuged (3500 rotations per minute, 5 minutes). The supernatant and pellet fractions were kept for further analysis. NaOH at concentrations of 0.2 N (200 μl) and 1 N (30 μl) were added to the pellet and supernatant, respectively, which were then boiled for 5 minutes and cooled to room temperature.

The protein content was determined for the pellet and supernatant samples. Degradation was calculated as the percentage of total protein (total=supernatant+pellet) present in the supernatant.

Determination of Protein Content:

Protein concentration was determined according to the modification described in Markwell et al. [Anal Biochem 1978, 87:206-210] of the method of Lowry et al. [J Biol Chem 1951, 193:265-275], using fibrinogen as standard, unless otherwise indicated.

Cell Adhesion Assay:

Human gingival fibroblasts were labeled with [³H]-thymidine (1 microcurie/ml in culture medium) for 48 hours, then harvested with trypsin, washed in culture medium (Alpha Minimum Essential Medium) supplemented with 12% fetal calf serum (FCS) and then resuspended, at a concentration of 30,000 cells/ml, either in culture medium or in culture medium supplemented with 12% FCS. To determine the radioactivity/cell ratio, 1 ml of cell suspension was digested with 0.5 N NaOH, and the level of radioactivity was measured in a TRI-CARB 1900 CA β-counter (Lumitron). 1 ml of cell suspension (with or without FCS) was then plated in each well. Cells were allowed to attach for 24 hours, and unattached cells were then carefully washed out with PBS. 1 ml of 0.5N NaOH was added to each well, and the radioactivity in each well was measured in a β-counter.

Cell Proliferation Assay:

Thin fibrin matrices were produced by casting 100 μl of freshly polymerized fibrin on 13 mm diameter cover slides. Following crosslinking the fibrin matrices attached to the slides were thoroughly rinsed in PBS. Two thousand human gingival fibroblast cells were seeded on each slide and cultured in culture medium (Alpha Minimum Essential Medium) supplemented with 12% FCS for 24 and 72 hours, at which times the experiment was terminated by fixing the cell in 1.5% paraformaldehyde. The slides were then washed in water, stained with hematoxylin, dehydrated, and mounted on histological slides. The number of cells was counted in 3 randomly selected fields of 5.06 mm² using an optical grid (CPLW10*/18, Zeiss, Germany). Three cover slides were assayed for each type of fibrin matrix at each time point. The mean number of cells/mm² of matrix was calculated.

### Example 1

### Injectable Crosslinked Fibrinogen (CL-Fibrinogen)

Fibrinogen was crosslinked as described hereinabove, by incubating 3 mg/ml fibrinogen in a solution comprising 70% ethanol and 1% ribose for 5 or 11 days.

Samples were subjected for 6 hours to a trypsin degradation assay, as described hereinabove, except that for some samples a higher concentration of trypsin was used.

As shown in FIG. 1, fibrinogen incubated with ethanol and ribose for 11 days is more resistant to degradation by trypsin than is fibrinogen incubated for 5 days.

As is further shown in FIG. 1, the maximal degradation rate was achieved at a trypsin concentration of 2000 units/ml.

Based on the results presented in FIG. 1, it was determined that a trypsin concentration of 1000 units/ml provides optimal sensitivity to the degree of crosslinking, and this concentration was used in the following experiments.

In order to further characterize the effect of incubation time on degradation, additional degradation assays were performed using 1000 units/ml trypsin and various degradation times.

As shown in FIG. 2, fibrinogen incubated with ethanol and ribose for 5 days was fully degraded after 6 hours, whereas fibrinogen incubated for 11 days was fully degraded only after 24 hours. The greatest difference in degradation was observed at 6 hours.

In order to determine the effect of ethanol concentration on crosslinking of fibrinogen, 1.5 ml of a 10 mg/ml fibrinogen solution was mixed with ethanol so as to give a final concentration of 50, 60, 70 or 85% ethanol, and ribose was added at a final concentration of 1%. Depending on the concentration of ethanol used, the final concentration of fibrinogen in these solutions was 3, 3, 2.75 and 1.5 mg/ml, respectively. The solutions were incubated for 11 days at 37° C., and the obtained CL-fibrinogen was then subjected to a trypsin assay.

As shown in Table 1 below, the lowest level of degradation was observed for 70% ethanol. As further shown, the concentration of ethanol had little effect on the yield.

These results indicate that 70% ethanol provides the greatest degree of crosslinking.

TABLE 1
Degradation and yield of CL-fibrinogen
at various ethanol concentrations

| | % Degradation | | |
|---|---|---|---|
| ethanol | 2 h | 6 h | Yield |
| 50% | 43.5 | 62.9 | 81% |
| 60% | 42.3 | 56.5 | 74% |
| 70% | 23.3 | 29.7 | 73% |
| 85% | 33.3 | 38.7 | 80% |

### Example 2

### Crosslinked Fibrin (CL-Fibrin) Matrix

Fibrinogen solutions at a concentration of 50 mg/ml of PBS were mixed with an equal volume of a solution of 5 units/ml thrombin.

Following 1 hour incubation at 37° C., a hydrogel matrix consisting of fibrin fibers was formed. The mechanical properties of the matrix allowed the matrix to be manipulated and transferred to a container consisting of 70% ethanol at room temperature. The ethanol facilitated sterilization and preparation for the crosslinking process.

In order to characterize the degree of crosslinking, fibrin matrices in the form of a membrane were incubated for 0, 3, 6 and 11 days in a solution of 90% ethanol and 1% ribose were washed in PBS and subjected to a trypsin degradation assay using a concentration of 0.25% trypsin. At time points of 0, 2, 4, 6 and 9 hours, samples were centrifuged to obtain a pellet, which was digested with 0.5 N NaOH. Protein concentration was then determined using a BCA kit (Pierce) according to the manufacturer's instructions.

As shown in FIG. 3, at 9 hours, the non-crosslinked fibrin and fibrin crosslinked for 3 days was completely degraded, fibrin crosslinked for 6 days was mostly degraded, and fibrin crosslinked for 11 days was not degraded at all. At 2 hours, fibrin crosslinked for 6 and 11 day was not degraded at all, and fibrin crosslinked for 3 days and non-crosslinked fibrin were respectively 17% and 60% degraded. At 6 hours, more than 80% of non-crosslinked fibrin and fibrin crosslinked for 3 days were degraded, whereas only 22% of fibrin crosslinked for 6 days was degraded.

These results indicate that the resistance to proteolytic degradation of fibrin matrices can be gradually increased by prolonging the incubation time in the crosslinking solution.

It was observed that the increase in resistance to proteolysis is accompanied by a change in color from whitish to yellow, along with an increase in opacity. Therefore, fibrin matrices having a volume of approximately 60 μl were prepared and incubated in crosslinking solution described above in 96-well ELISA plates. The resulting matrices were of a cylindrical shape, filling the space of the wells. The optical density (O.D.) of the matrices was then measured by an ELISA reader at 341 nm, following 0, 3, 6 and 11 days of incubation in crosslinking solution. The change in O.D. represents the change in turbidity and color of the matrix, which in turn represents the degree of crosslinking. The control is fibrin matrices following incubation for various time periods without crosslinking solution.

As shown in FIG. 4, the O.D. of fibrin matrices gradually increased over time.

In order to determine the effect of ribose concentration on the degree of crosslinking, fibrin matrices were prepared in 96-well ELISA plates and incubated in crosslinking solutions comprising 90% ethanol and 0 (control), 0.1, 0.25, 0.5, 1 or 2% ribose. The degree of crosslinking was characterized 1 hour and 1, 3, 5, 6, 7 and 11 days thereafter by measuring the O.D. in each well. Eight wells were used for each concentration and the experiment was repeated 3 times.

As shown in FIG. 5, the fibrin matrices incubated in crosslinking solutions containing ribose exhibited an almost linear increase in their O.D. over time. The increase in O.D. did not level off after 11 days of incubation, suggesting that further crosslinking can be obtained.

As further shown in FIG. 5, ribose concentrations between 0.1-0.5% had similar effects, conferring an approximately 3-fold increase in the O.D. over 11 days of incubation. Ribose concentrations of 1% and 2% resulted in increases of almost 4-fold in the O.D.

No significant differences were observed between the effect of 1% and 2% ribose, indicating that a ribose concentration of 1% was sufficient for optimal fibrin crosslinking.

In order to determine the effect of organic solvent concentration on the degree of crosslinking, fibrin matrices were prepared in 96-wells ELISA plates and incubated in crosslinking solutions comprising 50%, 70%, 90% or 100% ethanol, to which an additional 1% ribose was added. The degree of crosslinking was characterized 1 hour and 1, 2, 5, 7, 8, 9, 11 and 13 days thereafter by measuring the O.D. in each well. Five wells were used for each concentration and the experiment was repeated 3 times.

As shown in FIG. 6, incubation with ethanol resulted in a progressive increase in the O.D. of fibrin matrices, which increased by approximately 2.5-fold following 11 days of incubation with 50% or 70% ethanol, and by approximately 4-fold following 11 days of incubation with 90% or 100% ethanol.

The above results indicate that a solution comprising 90% ethanol (with 10% PBS) provides optimal crosslinking for fibrin matrices.

In view of the above results, a crosslinking solution of 90% ethanol and 10% PBS, to which 1% ribose was added, was used for further experiments with fibrin matrices. Such a solution is referred to herein as optimal crosslinking solution (OCS).

In order to determine the effect of fibrin concentration on the degree of crosslinking, fibrin matrices having a volume of approximately 60 μl and a fibrin concentration of 25, 50 or 100 mg/ml were prepared as described hereinabove and incubated in OCS for up to 14 days. Matrices of the same fibrin content incubated in a 90% ethanol solution with no ribose served as controls. The degree of crosslinking was characterized 1 hour and 1, 2, 5, 6, 7, 11, 13 and 14 days thereafter by measuring the O.D. at 341 nm in each well. Five wells were used for each concentration.

As shown in FIG. 7, increased fibrin concentration resulted in greater increases of O.D.

These results indicate that higher fibrin concentrations result in a higher degree of fibrin crosslinking.

A fibrin matrix comprising 50 mg/ml fibrin (25 mg fibrin in a volume of 0.5 ml) was crosslinked with OCS and subjected to a trypsin degradation assay with 0.25% trypsin, as described hereinabove. Non-crosslinked matrices served as controls. Samples were harvested after 1, 2, 4, 6, 24, 48, and 100 hours of proteolytic digestion and the degree of degradation was determined using the abovementioned BCA method.

As shown in FIG. 8, non-crosslinked matrices were completely degraded after 6 hours of incubation in trypsin, whereas by that time only 8.5% of the crosslinked matrices were degraded. Crosslinked matrices were completely degraded after 100 hours of digestion. The degradation half-life of the crosslinked matrices was 48 hours, whereas the half-life for non-crosslinked matrices was 2 hours, indicating that crosslinking provided a 24-fold increase in the resistance to degradation.

### Example 3

### Degradation of Injectable CL-Fibrin and Injectable CL Fibrinogen

Injectable CL-fibrin (at a concentration of 10 mg/ml in the crosslinking process) and injectable CL-fibrinogen (at a concentration of 5 mg/ml in the cross-linking process) were prepared as described in the Materials and Methods section. Crosslinking for both CL-fibrin and CL-fibrinogen was performed by incubation for 11 days in a solution of 70% ethanol and 30% PBS, with 1% ribose added. Both of the crosslinked proteins were subjected to the trypsin degradation assay for 2 and 6 hours.

As shown in Table 2, both the resistance to degradation and the yields obtained were similar for injectable CL-fibrin and injectable CL-fibrinogen.

TABLE 2
Degradation and yield for injectable CL-fibrin
and injectable CL-fibrinogen

| | % Degradation | | |
|---|---|---|---|
| | 2 hours | 6 hours | Yield |
| CL-Fibrin | 27.4 | 34.8 | 87% |
| CL-Fibrinogen | 22.6 | 37.5 | 84% |

### Example 4

### Final Protein Concentrations of Injectable CL-Fibrin and Injectable CL Fibrinogen

Injectable CL-fibrin (at a concentration of 10 mg/ml in the crosslinking process) and injectable CL-fibrinogen (at a concentrations of 2.5 and 5 mg/ml in the cross-linking process) were prepared as described in the Materials and Methods section. Crosslinking for both CL-fibrin and CL-fibrinogen was performed by incubation for 11 days in a solution of 70% ethanol and 30% PBS, with 1% ribose added. The crosslinked proteins were washed and centrifuged at a force of 1200 g for 10 minutes. The pellets of the packed crosslinked protein were collected, their volume and protein content were determined, and the final protein concentration of the packed products was calculated.

As shown in Table 3, the final protein concentration of injectable CL-fibrin is higher than that of injectable CL-fibrinogen. As using a higher fibrinogen concentration during crosslinking resulted in only a slightly higher final protein concentration of injectable CL-fibrinogen, it is unlikely that the higher concentration of protein in injectable CL-fibrin is due solely to the higher initial protein concentration which was used.

TABLE 3
Protein concentrations for injectable CL-fibrin
and injectable CL-fibrinogen

| | Initial protein | Product protein |
|---|---|---|
| | concentration | concentration |
| | (mg/ml) | (mg/ml) |
| CL-Fibrin | 10 | 30.0 |
| CL-Fibrinogen | 2.5 | 16.3 |
| CL-Fibrinogen | 5 | 19.0 |

### Example 5

### Microscopic Structure of Injectable CL-Fibrin and Injectable CL-Fibrinogen

Injectable CL-fibrin and injectable CL-fibrinogen were prepared as described in Example 3, and examined by Environmental Scanning Electron Microscope (ESAM).

As shown in FIGS. 9A and 9B, large smooth fibrils of injectable CL-fibrin were observed, having a diameter of 3-5 microns.

In contrast, as shown in FIGS. 10A and 10B, injectable CL-fibrinogen appeared as a porous amorphous material. As shown in FIG. 10B, the material appeared as an aggregate of numerous spheres having a diameter of approximately 0.25 microns.

### Example 6

Comparison of Degradation of Fibrin Matrix Crosslinked with Ribose and Fibrin Matrix Crosslinked by Transglutaminase (Factor XIIIa)

Fibrin clots formed in vivo following wounding of blood vessels are immediately crosslinked by the enzyme transglutaminase (factor XIIIa) which is formed from the zymogen factor XIII after cleavage of its propeptide by thrombin. Transglutaminase forms covalent crosslinks between lysine and glutamine that confer mechanical stability and proteolytic resistance to the clot.

To test whether fibrin matrices prepared according to embodiments of the present invention exhibit more proteolytic resistance than do naturally formed fibrin matrices, fibrin matrices 0.2 ml in volume with 10 mg/ml fibrin were prepared and divided into 3 groups. The first experimental group was crosslinked by incubation in the OCS described hereinabove for 11 days. The second experimental group was crosslinked with transglutaminase (0.25 units/ml) for 1 hour according to the procedure described in Sun et al. [Biopolymers 2005, 77:257-263]. The third group served as control and was not crosslinked. The samples from each group were then subjected to a trypsin degradation assay. Samples were harvested at 0, 2, 4, 6 and hours following trypsin addition.

As shown in FIG. 11, fibrin matrices crosslinked with ethanol and ribose (OCS) were more resistant to degradation than were fibrin matrices crosslinked with transglutaminase. The degradation half-lives of the non-crosslinked control matrices, the transglutaminase-crosslinked matrices and the OCS-crosslinked matrices were 2, 4 and 8 hours, respectively. By 6 hours, the non-crosslinked matrices and the transglutaminase-crosslinked matrices were completely degraded, whereas only the OSC-crosslinked matrices only 16.8% degraded.

These results indicate that the ribose crosslinking process is considerably more potent that transglutaminase crosslinking process in conferring proteolytic resistance to fibrin matrices.

### Example 7

### Support of Cell Attachment and Proliferation by CL-Fibrin Matrix

The formation of new intramolecular and intermolecular covalent bonds and glycosylation may change the biological properties of the crosslinked proteins described herein. To test this possibility, the capacity of CL-fibrin matrices to support cell attachment and proliferation was determined.

Fibrin matrices having a volume of 0.3 ml were prepared in the form of a membrane, as described above, in 16 wells of 24-well plates. Half of the matrices were crosslinked in OCS for 11 days and then washed thoroughly in PBS. The second half was left untreated. The remaining 8 wells that did not contain fibrin matrices served as controls. The fibrin matrices and control wells were then subjected to cell adhesion assays, as described hereinabove in the Materials and Methods section.

As shown in FIG. 12, cell adhesion was 30% to 50% lower in crosslinked fibrin matrices than in both non-crosslinked matrices and polystyrene surfaces, both in the presence and in the absence of FCS.

These results indicate that the crosslinking process described herein blocks cell attachment sites on fibrin, possibly interfering with binding of fibronectin, the major serum attachment protein to fibrin.

Fibrin matrices were produced by casting fibrin on cover slides, in order to perform the cell proliferation assay described hereinabove in the Materials and Methods section. Some of the slide-attached fibrin matrices were crosslinked in OCS and the rest were incubated in PBS for the same period of time. The slides were then plated with cells as described hereinabove.

The number of cells counted 1 day (24 hours) after plating represent the number of attached cells to each type of matrix.

As shown in FIG. 13, the number of cells/mm² observed after 1 day was 1.57-fold higher on the non-crosslinked matrices than in the crosslinked matrices.

This result confirms the above results obtained with the cell adhesion assay, indicating that both methodologies for cell number determination are reliable.

As further shown in FIG. 13, the number of cells per mm² 72 hours after plating was 2.3-fold the number counted after 24 hours on crosslinked fibrin, and 1.8-fold the number counted after 24 hours on non-crosslinked fibrin.

These results indicate that crosslinked fibrin matrices have a higher capacity to support cell proliferation than do non-crosslinked fibrin matrices.

### Example 8

### Support of Cell Attachment and Proliferation by CL Fibrinogen Matrix

The capacity of CL-fibrin matrices to support cell attachment and proliferation is determined as described hereinabove for CL-fibrin in Example 7.

Crosslinked fibrinogen matrices are prepared in wells of 24-well plates by being crosslinked in a solution comprising ethanol (e.g., 70% ethanol) and ribose (e.g., 1% ribose) for several days (e.g. 11 days) and then washed thoroughly in PBS. Crosslinked and/or non-crosslinked fibrin matrices are prepared as described hereinabove in Example 7 for comparison. Additional wells which do not contain any protein matrices serve as controls.

The fibrinogen matrices and control wells are then subjected to cell adhesion assays, as described hereinabove in the Materials and Methods section.

Crosslinked fibrinogen matrices are prepared by being crosslinked in a solution comprising ethanol (e.g., 70% ethanol) and ribose (e.g., 1% ribose) over the surface of a cover slide so as to form a layer of precipitated fibrinogen on the surface of the cover slide. The fibrinogen is incubated in the solution for several days (e.g. 11 days), so as to form a crosslinked fibrinogen matrix on the cover slide, in order to perform the cell proliferation assay described hereinabove in the Materials and Methods section. Crosslinked and/or non-crosslinked fibrin matrices are prepared as described hereinabove and cast on cover slides for comparison. The slides are then plated with cells as described hereinabove.

The number of cells counted 1 day after plating represents the number of attached cells to each type of matrix.

The number of cells counted 3 days after plating indicates the amount of proliferation since day 1.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1-87. (canceled)

88. A composition-of-matter comprising fibrinogen being crosslinked with at least one reducing sugar.

89. The composition-of-matter of claim 88, wherein said reducing sugar is ribose.

90. The composition-of-matter of claim 88, characterized by a structure comprising an aggregation of microparticles.

91. The composition-of-matter of claim 88, being in an injectable form.

92. The composition-of-matter of claim 88, having a concentration of fibrinogen in a range of 1 mg/ml to 100 mg/ml.

93. A process for producing a composition-of-matter comprising fibrinogen being crosslinked with at least one reducing sugar, the process comprising reacting fibrinogen with said at least one reducing sugar in a crosslinking solution which comprises said reducing sugar and a polar organic solvent.

94. The process of claim 93, wherein a concentration of said reducing sugar is in a range of 0.1% to 6%.

95. The process of claim 93, wherein fibrinogen is insoluble in said polar organic solvent, and said process further comprises precipitating said fibrinogen in a solution comprising said polar organic solvent.

96. The process of claim 93, wherein a concentration of said polar organic solvent is in a range of 50% to 100% per volume of the crosslinking solution.

97. The process of claim 93, further comprising converting the composition-of-matter to an injectable form, said converting comprising particulation of the composition-of-matter into particles of a size sufficiently small so as to be suitable for injection.

98. A composition-of-matter obtainable by the process of claim 93.

99. The composition-of-matter of claim 88, further comprising a pharmaceutically active agent being contained within the composition-of-matter or on a surface of the composition-of-matter.

100. A pharmaceutical, cosmetic or cosmeceutical composition comprising the composition-of-matter of claim 88 and a pharmaceutically, cosmetically or cosmeceutically acceptable carrier.

101. A pharmaceutical, cosmetic or cosmeceutical composition comprising the composition-of-matter of claim 98 and a pharmaceutically, cosmetically or cosmeceutically acceptable carrier.

102. A method of treating a medical disorder or a cosmetic disorder characterized by a tissue damage in a subject in need thereof, the method comprising administering to the subject a therapeutically effective amount of the composition-of-matter of any of claim 88.

103. A method of treating a medical disorder or a cosmetic disorder characterized by a tissue damage in a subject in need thereof, the method comprising administering to the subject a therapeutically effective amount of the composition-of-matter of any of claim 98.

104. A method of performing a procedure selected from the group consisting of tissue regeneration, wound healing, tissue engineering, drug delivery and tissue augmentation in a subject in need thereof, the method comprising administering to the subject the composition-of-matter of claim 88.

105. A method of performing a procedure selected from the group consisting of tissue regeneration, wound healing, tissue engineering, drug delivery and tissue augmentation in a subject in need thereof, the method comprising administering to the subject the composition-of-matter of claim 98.

106. The method of claim 104, wherein said composition-of-matter is administered to said subject by implantation.

107. The method of claim 104, wherein said composition-of-matter is administered to said subject by injection.

108. A medical device composed of, or comprising, the composition-of-matter of claim 88.

109. A kit for generating the composition-of-matter of claim 88, the kit comprising
(i) fibrinogen; and
(ii) a reducing sugar.

110. The kit of claim 109, further comprising a polar organic solvent.

111. A composition-of-matter comprising fibrin being crosslinked with at least one reducing sugar.

112. The composition-of-matter of claim 111, wherein said reducing sugar is ribose.

113. The composition-of-matter of claim 111, exhibiting a resistance to proteolytic degradation which is at least 20% higher than that of Factor XIIIa-crosslinked fibrin.

114. The composition-of-matter of claim 111, being in an injectable form.

115. The composition-of-matter of claim 111, having a concentration of fibrin in a range of 10 mg/ml to 150 mg/ml.

116. A process for producing a composition-of-matter comprising fibrin being crosslinked with at least one reducing sugar, the process comprising reacting fibrin with said at least one reducing sugar in a crosslinking solution which comprises said reducing sugar and a polar organic solvent.

117. The process of claim 116, wherein a concentration of said reducing sugar is in a range of 0.1% to 6%.

118. The process of claim 116, further comprising, prior to reacting fibrin with said reducing sugar, reacting fibrinogen with thrombin so as to obtain fibrin.

119. The process of claim 116, wherein a concentration of said polar organic solvent is at least 50% per volume of the crosslinking solution.

120. The process of claim 116, further comprising converting the composition-of-matter to an injectable form, said converting comprising particulation of the composition-of-matter into particles of a size sufficiently small so as to be suitable for injection.

121. A composition-of-matter comprising fibrin obtainable by the process of claim 116.

122. The composition-of-matter of any of claim 111, further comprising a pharmaceutically active agent being contained within the composition-of-matter or on a surface of the composition-of-matter.

123. A pharmaceutical, cosmetic or cosmeceutical composition comprising the composition-of-matter of claim 111 and a pharmaceutically, cosmetically or cosmeceutically acceptable carrier.

124. A pharmaceutical, cosmetic or cosmeceutical composition comprising the composition-of-matter of claim 121 and a pharmaceutically, cosmetically or cosmeceutically acceptable carrier.

125. A method of treating a medical disorder or a cosmetic disorder characterized by a tissue damage in a subject in need thereof, the method comprising administering to the subject a therapeutically effective amount of the composition-of-matter of claim 111.

126. A method of treating a medical disorder or a cosmetic disorder characterized by a tissue damage in a subject in need thereof, the method comprising administering to the subject a therapeutically effective amount of the composition-of-matter of claim 121.

127. A method of performing a procedure selected from the group consisting of tissue regeneration, wound healing, tissue engineering, drug delivery, and tissue augmentation in a subject in need thereof, the method comprising administering to the subject the composition-of-matter of claim 111.

128. A method of performing a procedure selected from the group consisting of tissue regeneration, wound healing, tissue engineering, drug delivery, and tissue augmentation in a subject in need thereof, the method comprising administering to the subject the composition-of-matter of claim 121.

129. The method of claim 127, wherein said composition-of-matter is administered to said subject by implantation.

130. The method of claim 127, wherein said composition-of-matter is administered to said subject by injection.

131. A medical device composed of, or comprising, the composition-of-matter of claim 111.

132. A kit for generating a composition-of-matter comprising crosslinked fibrin, the kit comprising:
(i) fibrinogen;
(ii) thrombin; and
(iii) a reducing sugar.

133. The kit of claim 132, further comprising a polar organic solvent.
